(19) United States
(12) Patent Application Publication
(10) Pub. No.: US 2021/0315559 A1
(43) Pub. Date: Oct. 14, 2021

(54) MANIPULATING DEVICE WITH TUBE WALL FORMATIONS

(71) Applicants: Hagay Drori, Tel Aviv (IL); Hamid Sharim, Kokhav Yair (IL); Yuri Berlin, Kiryat Bialik (IL); Tovy Sivan, Kfar Saba (IL)

(72) Inventors: Hagay Drori, Tel Aviv (IL); Hamid Sharim, Kokhav Yair (IL); Yuri Berlin, Kiryat Bialik (IL); Tovy Sivan, Kfar Saba (IL)

(21) Appl. No.: 15/556,391

(22) PCT Filed: Mar. 6, 2016
(86) PCT No.: PCT/IB2016/051261

§ 371 Date: Dec. 4, 2017

Related U.S. Application Data
(60) Provisional application No. 62/129,806, filed on Mar. 7, 2015.

(51) Int. Cl. A61B 17/00; A61B 17/02; A61B 17/29

(52) U.S. Cl. CPC A61B 17/00234; A61B 17/0218; A61B 2017/00314; A61B 2017/00526; A61B 17/29

(57) ABSTRACT

A manipulating device includes a hollow tube having a tube wall formed with distal cutouts that define at least one deformable member in the tube wall and proximal cutouts that define an actuator in the tube wall connected to the at least one deformable member. Movement of the actuator deforms the at least one deformable member from a first orientation to a second orientation.

### FIELD OF THE INVENTION

The present invention relates generally to manipulating medical devices, and particularly to a tube with a deformable portion connected to a manipulating portion (actuator).

### BACKGROUND OF THE INVENTION

There are many deformable and expandable cannulas designed for surgical use. In many surgical applications, it is desirable to use surgical tools that are small so that access to the targeted tissue site can be achieved using minimally invasive procedures. In addition, it is desirable to use surgical tools that allow passage of other surgical instruments and medical devices through a lumen formed in the tool.

However, prior art expandable cannulas employ some kind of actuator that surrounds the cannula or enters a lumen of the cannula, such as a surrounding sheath, sleeve, shaft, distal support, internal rod, balloon, hinge, and many more. These limitations require larger devices and cannot allow the passage of surgical tools through the lumen at the same time the actuator is deployed.

### SUMMARY OF THE INVENTION

The present invention seeks to provide an improved manipulating device which is a steerable tube, with an integrally formed deformable portion and manipulating portion (actuator), as described in more detail further below. The device is a unitary (one-piece) hollow tube (e.g., hollow lumen through the entire length of the tube) that can deform (e.g., bend and/or expand and contract) and allow the passage of surgical tools, fluids or other materials through the lumen while at the same time deforming another portion of the tube to any desired deformation.

The tube of the invention encompasses, without limitation, a needle (having a sharp tip for piercing tissue), a cannula, a guidewire, a catheter etc., all with a hollow lumen. The tube can be formed, without limitation, as a needle, cannula, guidewire, forceps, punch, scissors, drill/burr, shaver, RF cautery, electrodes, and many more.

In contrast with the prior art, in the present invention forces are transmitted through the tube's wall with a compliant working tip mechanism. The cannula, working tip and manipulating mechanism are formed from a single tube.

The device has many applications, such as but not limited to, epidural injections, percutaneous, endoscopic and laparoscopic therapeutic or diagnostic procedures, Natural Orifice Transluminal Endoscopic Surgery (NOTES, e.g. Endo-Nasal Skull Base Surgery (ENSBS)), endovascular procedures, bronchoscopies, orthopedic procedures as meniscus repair, carpal tunnel repair, spinal procedures, Transurethral Prostate Surgery, and biopsies.

For example, spinal stenosis is a narrowing of the open spaces in the spine, often caused by wear-and-tear changes associated with aging. This narrowing puts pressure on the spinal cord, causing back pain, leg pain and other symptoms. Epidural injections are one of the most commonly performed interventions in the United States prescribed to treat pain in the early stages of spinal stenosis, as an alternative to surgery. However, epidural injections, even if they have some chemical benefits, have risks. Ligamentum flavum is a ligament posterior to the dural sac which is punctured by epidural steroids injections. Thickening of the ligamentum flavum is one of the causes of lumbar spinal stenosis, and is due to buckling of the ligamentum flavum into the spinal canal secondary to disc degeneration. The ligamentum flavum is usually resected during spinal surgery for spinal stenosis. Preservation of the ligamentum flavum was suggested to reduce postoperative scarring after spinal disc surgery, as an intact ligament which protects the dural sac from adhesions.

In contrast to the prior art, the present invention provides an expandable device intended to work in adjacent to an epidural needle for reversal of the buckling of the ligamentum flavum during the epidural injection procedure, to expand the spinal canal space while preserving the ligamentum flavum and to increase the short and long-term benefit of epidural injections, thereby improving the benefit of the treatment. The expanded portion provides tension around the epidural needle to adjust the position of the ligamentum flavum out of the canal.

Another example is discectomy, which is a technique for the decompression of the lumbar disc space and removal of nucleus pulposus for patients with lumbar disc herniation.

Conventional (open) microdiscectomy is the most frequently performed surgery for patients with sciatica due to lumbar disc herniation. Various minimally invasive surgical techniques are seeing increasing popularity, such as: microendoscopic discectomy (MED), and percutaneous microdiscectomy.

However, in the prior art, endoscopic discectomy is not universally accepted, inter alia, due to the difficult skill set required of the surgeon to perform the delicate maneuvers. The device of the invention provides a solution to the difficulty of performing discectomy, wherein the deformable portion of the tube expands outwards to create a nerve protector accessory (“protector net”) for endoscopic lumbar decompression. The expanded portion provides a safe working zone, by protecting the nerves and dural sac.

There is thus provided in accordance with an embodiment of the present invention a manipulating device including a hollow tube having a tube wall formed with distal cutouts that define at least one deformable member in the tube wall and proximal cutouts that define an actuator in the tube wall connected to the at least one deformable member, wherein movement of the actuator deforms the at least one deformable member from a first orientation to a second orientation. The at least one deformable member includes jaws, wherein in the second orientation the jaws are separated from each other by a larger gap than in the first orientation.

It is noted throughout that the terms “distal” and “proximal” are relative and mean that one portion or element is more distal than the other element or portion. The distal portion or element does not have to be at the most distal end of the device and the proximal portion or element does not have to be at the most proximal end of the device. Rather the distal portion or element can be anywhere along the device and is simply closer to the most distal end of the device than the proximal portion or element.

In accordance with an embodiment of the present invention the at least one deformable member includes a stationary jaw and a movable jaw, wherein in the second orientation the jaws are separated from each other by a larger gap than in the first orientation.

In accordance with an embodiment of the present invention the at least one deformable member includes a plurality of deformable members connected by linkage members defined by other cutouts formed in the tube wall.

In accordance with an embodiment of the present invention at least one of the linkage members is connected to the actuator.

In accordance with an embodiment of the present invention the linkage members extend from one portion (e.g., a distal portion) of the deformable members towards another (e.g., a proximal portion) of the tube wall.

In accordance with an embodiment of the present invention at least one of the deformable members extends proximally from a distal portion of one of the linkage members and then curves back to a distal portion of another of the linkage members. The deformable members and linkage members may be connected in other ways as well.

In accordance with an embodiment of the present invention the at least one deformable member expands outwards or inwards in the second orientation.

In accordance with an embodiment of the present invention the at least one deformable member in the second orientation may serve as a retractor, for example, to retract nerves out of the deformable member so as to protect them from instruments that may enter in that area (e.g., grasper, punch, burr, etc.). As another example, the deformable member in the second orientation may form a protection shroud for objects located inwards of the deformable members.

In accordance with an embodiment of the present invention the at least one deformable member includes cutting surfaces.

In accordance with an embodiment of the present invention the at least one deformable member includes a grasping element.

In accordance with an embodiment of the present invention the at least one deformable member includes an electrode.

In accordance with an embodiment of the present invention the actuator includes a handle interface portion, for example, for connecting to a handle or other tool, such as by screws, pins, bonding, welding or any other method.

In accordance with an embodiment of the present invention the tube wall has an additional cutout in which the actuator is movable.

There is also provided in accordance with an embodiment of the present invention a method for making a manipulating device including forming at least one deformable member and an actuator connected to the at least one deformable member by making cutouts in a wall of a hollow tube, the cutouts defining outlines of the at least one deformable member and the actuator.

### BRIEF DESCRIPTION OF DRAWINGS

The present invention will be understood and appreciated more fully from the following detailed description, taken in conjunction with the drawings in which:

FIGS. 1A and 1B are simplified pictorial illustrations of a manipulating device, constructed and operative in accordance with an embodiment of the present invention, wherein in FIG. 1A the device is not expanded (not deformed) and in FIG. 1B the device is expanded (deformed);

FIG. 2 is a simplified pictorial illustration of a manipulating device with more than one deformable portion, in accordance with another embodiment of the present invention;

FIGS. 3A, 3B and 3C are simplified front, side and rear view illustrations, respectively, of a manipulating device with a distal ring that does not deform but deflects, in accordance with another embodiment of the present invention;

FIGS. 3D and 3E are simplified illustrations of the manipulating device of FIG. 3A before and after deflection of the distal ring;

FIGS. 4A, 4B and 4C are simplified pictorial, front and side view illustrations, respectively, of a manipulating device with grasper or cutter jaws, in accordance with another embodiment of the present invention;

FIGS. 5A, 5B and 5C are simplified front, side and rear view illustrations, respectively, of a manipulating device with deformable struts, in accordance with another embodiment of the present invention;

FIGS. 5D-5E, 5F-5G and 5H-5I are three pairs of illustrations, showing orientations of the deformable members and linkage members of the manipulating device of FIGS. 5A-5C, respectively, before and after deformation;

FIGS. 6A, 6B and 6C are simplified pictorial, front and side view illustrations, respectively, of a manipulating device, in accordance with another embodiment of the present invention;

FIGS. 6D and 6E are more detailed illustrations of the distal and proximal cutouts in the tube wall, respectively; and

FIGS. 7A-7B, 7C-7D, 7E-7F, 7G-7H, 7I-7J, 7K-7L and 7M-7N are pairs of illustrations, showing orientations of the deformable members and linkage members of manipulating devices of embodiments of the invention, respectively, before and after actuation of the actuator.

### DETAILED DESCRIPTION OF EMBODIMENTS

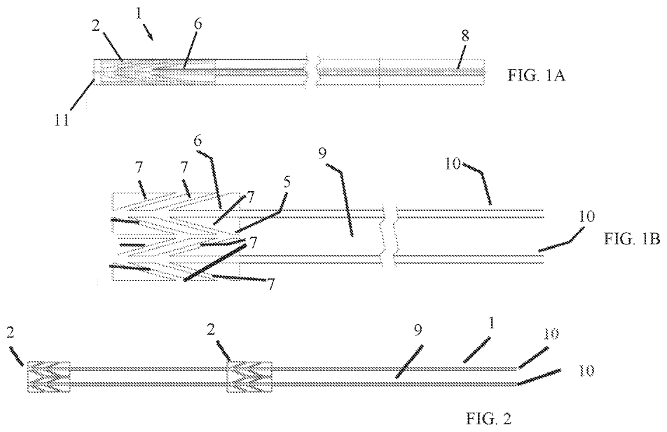

Reference is now made to FIGS. 1A and 1B, which illustrate a manipulating device 1, constructed and operative in accordance with an embodiment of the present invention. The manipulating device is a tube, cannula, catheter or needle (the terms being used interchangeably throughout). The manipulating device 1 has a deformable portion 2 and an actuator 8 formed as one integral, unitary piece from a single tube. The manipulating device may be used as a standalone device (which could be used alone or with other tools) or may be attached to another surgical tool, such as a laparoscopic, endoscopic, handheld, robotic or power tool, for example. The manipulating device may be, without limitation, a tool for grasping, dilating, tensioning, steering, adjusting, cavitating, cutting, stitching, stabilizing, etc., such as, without limitation, a tube, cannula, catheter, needle (straight/curved), rongeur, trocar, endoscope, stylet, etc. Attachment to the surgical tool may be achieved by laser welding, adhesives, solvents, mechanical attachment, or any other suitable method. The manipulating device may be attached to an RF ablation tool, wherein the deformable portion (or other portions of the manipulating device) serves as an electrode (e.g., monopolar, bipolar or combination thereof).

The device may be constructed from metal, polymer, super-elastic alloy, shape memory alloy, shape memory polymer, silicone, woven metal or polymeric wires and many other suitable materials.

The cannula has an outer diameter and a wall thickness; the internal diameter may or may not be concentric with the outer diameter (if not concentric, the tube will bend more easily at certain angles than other angles).

In the illustrated embodiment, the deformable portion 2 includes several struts attached to, and/or integral with each other, such as fixed struts 5, movable struts 6, and connecting struts 7. Each strut can have a different cross section, some struts can have similar cross sections, or all struts can have equal cross sections. The thickness and shape of the cross sections can be uniform or non-uniform.

The movable struts 6 may be connected to movable members 10 of the actuator 8. The fixed struts 5 may be connected to fixed members 9 of the actuator 8. The connecting struts 7 connect the fixed struts 5 to the movable struts 6. The connecting struts 7 may connect the movable struts 6 directly to the fixed member 9 (without a fixed strut 5), or alternatively may connect the fixed strut 5 directly to the movable member 10 (without a movable strut 6). The struts may be either straight or curved. All of the struts or part of the struts are deformable by application of force from the actuator 8.

In the illustrated embodiment, the deformable portion 2 includes a distal ring 11, which may be useful to protect elements like sensitive tissue (for example, the dural sac) during epidural insertion or insertion of the cannula into the ligamentum flavum. In other embodiments there is no distal ring. In this embodiment the distal ring also deforms; in other embodiments the distal ring does not deform during deformation of the struts.

In this embodiment, the deformable member 2 expands outwards in the deformed orientation, and forms a protection shroud for objects located inwards of the deformable members. Other non-limiting uses include cavitating, tensioning, adjusting, and/or dilating tissue.

FIG. 2 illustrates another manipulating device, constructed and operative in accordance with another embodiment of the present invention. This device differs from the device of FIGS. 1A-1B, in that there is more than one deformable portion 2, one proximal to the other and axially spaced from each other.

One method of using the above-described devices includes inserting the cannula into a procedure site, and deforming (e.g., expanding) the deformable portion 2 within tissue so as to distract, cavitate, tension, dilate, stitch, adjust, anchor, cut, pierce, grasp or protect tissues or other body structures for a surgical procedure. Afterwards, the deformable portion 2 may be returned to its original, undeformed state (e.g., contracted) or close to its original, undeformed state, and removed.

Optionally, the cannula may be covered with a sleeve (e.g., a polytetrafluoroethylene (PTFE) sleeve or similar), which may allow administration or removal of fluids or tissue.

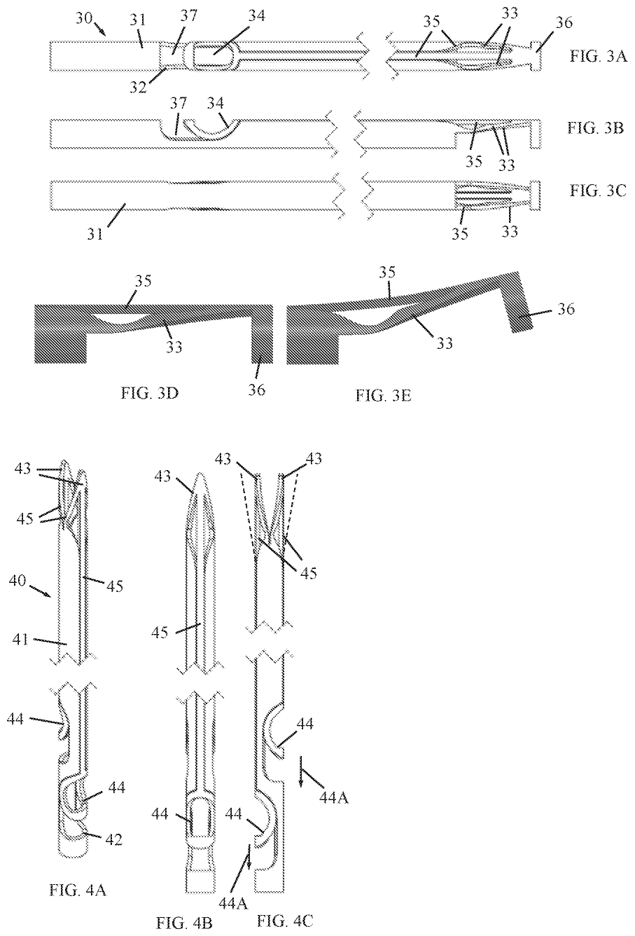

Reference is now made to FIGS. 3A-3E, which illustrate a manipulating device 30, in accordance with another embodiment of the present invention.

Manipulating device 30 includes a hollow tube 31 having a tube wall 32 (best seen in FIG. 3A) formed with distal cutouts that define one or more deformable members 33 in the tube wall 32 and proximal cutouts that define an actuator 34 in the tube wall 32 connected to deformable members 33. The deformable members 33 may be connected by linkage members 35 defined by other cutouts formed in tube wall 32. At least one of the linkage members 35 is connected to actuator 34, as seen in FIG. 3A. A distal ring 36 is located distally from the deformable members 33.

In accordance with an embodiment of the present invention the actuator 34 includes a handle interface portion, for example, for connecting to a handle or other tool, such as by screws, pins, bonding, welding or any other method, which makes it easy for the surgeon to move the actuator 34 back and forth (axially along the longitudinal axis of the device 30). The tube wall may have an additional cutout 37 (FIGS. 3A and 3B) in which actuator 34 is movable.

Movement of actuator 34 deforms deformable members 33 from a first orientation (FIGS. 3A-3D) in which the device is straight, to a second orientation (FIG. 3E) in which the deformable members 33 buckle and deform to one side and in which distal ring 36 also deflects to the side. It is noted that in this embodiment, distal ring 36 does not deform but only deflects. In this embodiment, the device is used as a steering device, wherein another surgical tool can be positioned in the inner diameter of the device (e.g., needle knife, grasper, light, viewing element, laser probe, suction tube, etc.).

Reference is now made to FIGS. 4A, 4B and 4C, which illustrate a manipulating device 40, in accordance with another embodiment of the present invention. This embodiment is similar to the embodiment of FIG. 3A, in that device 40 also includes a hollow tube 41 having a tube wall 42 formed with distal cutouts that define one or more deformable members 43 and proximal cutouts that define an actuator 44 in the tube wall 42 connected to deformable members 43. The deformable members 43 may be connected by linkage members 45 defined by other cutouts formed in tube wall 42. At least one of the linkage members 45 is connected to actuator 44, as seen in FIG. 4A. Some of the linkage members 45 may extend from a distal portion of the deformable members 43 towards a proximal portion of the tube wall 42.

This embodiment includes one or more actuators 44, (more than one actuator 44 is used in the illustrated embodiment), and each actuator 44 is formed from the tube wall. The deformable members 43 may form grasper or cutter jaws (in which case, the edges or other surface of the jaws are sharp). Each jaw is separately controlled by its own dedicated actuator 44. By pulling the actuator 44 proximally (as indicated by arrow 44A), the jaw is bent outwards (as indicated by the broken line). Accordingly, in one orientation (when deformed) the jaws are separated from each other by a larger gap than in another orientation (when undeformed). If only one actuator is provided or actuated, then one of the jaws is stationary and the other is movable; if two actuators are provided, then both jaws are movable.

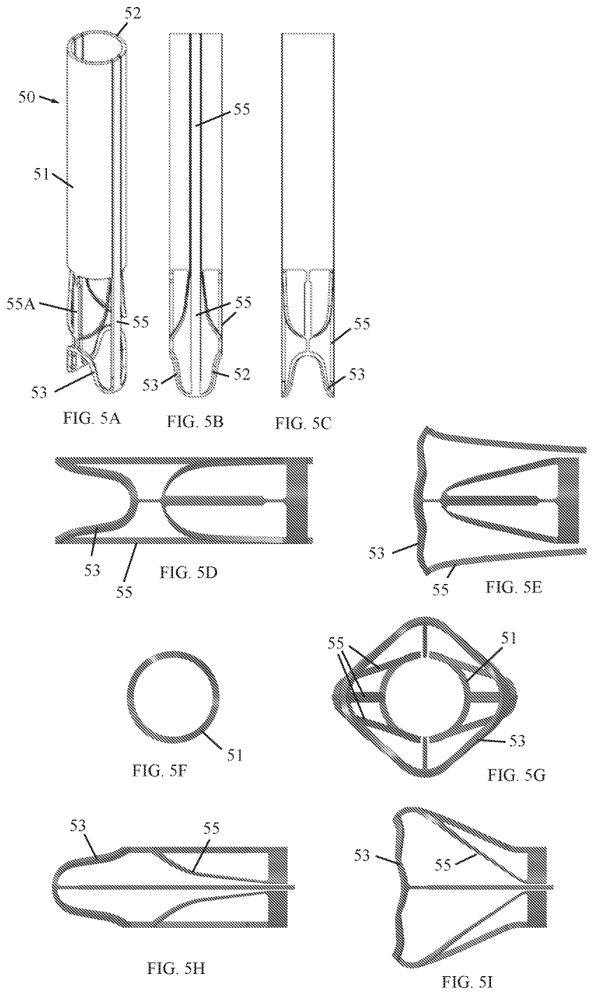

Reference is now made to FIGS. 5A-5C, which illustrate a manipulating device 50 with deformable struts, in accordance with another embodiment of the present invention.

Manipulating device 50 includes a hollow tube 51 having a tube wall 52 formed with distal cutouts that define one or more deformable members 53 in the tube wall 52. An actuator (not shown) is formed by proximal cutouts in the tube wall 52 (similarly to the previous embodiments) connected to deformable members 53. The deformable members 53 may be connected by linkage members 55 defined by other cutouts formed in tube wall 52.

As seen in FIG. 5B, some of the linkage members (designated by 55A) extend from a distal portion of the deformable members 53 towards a proximal portion of the tube wall 52. One or more of the deformable members 53 may extend proximally from a distal portion of one of the linkage members 55 and then curve back to a distal portion of another of the linkage members 55.

Reference is now made to FIGS. 5D-5E, 5F-5G and 5H-5I, which illustrate orientations of the deformable members 53 and linkage members 55 of the manipulating device 50, respectively, before and after deformation.

It is noted that some of the deformable members 53 may have different thicknesses than other deformable members 53 and/or some of the deformable members 53 may have varying thicknesses, that is, different thicknesses at different portions thereof. This feature provides the manipulating device with portions that are easier to bend or deform than other portions. For example, the thinner portions will bend or otherwise deform before the thicker portions start to bend or deform. As another example, the thinner portions will bend or otherwise deform at a greater amount than the thicker portions. The same holds true for linkage members 55: some of the linkage members 55 may have different thicknesses than other linkage members 55 and/or some of the linkage members 55 may have variable thicknesses, that is, different thicknesses at different portions thereof.

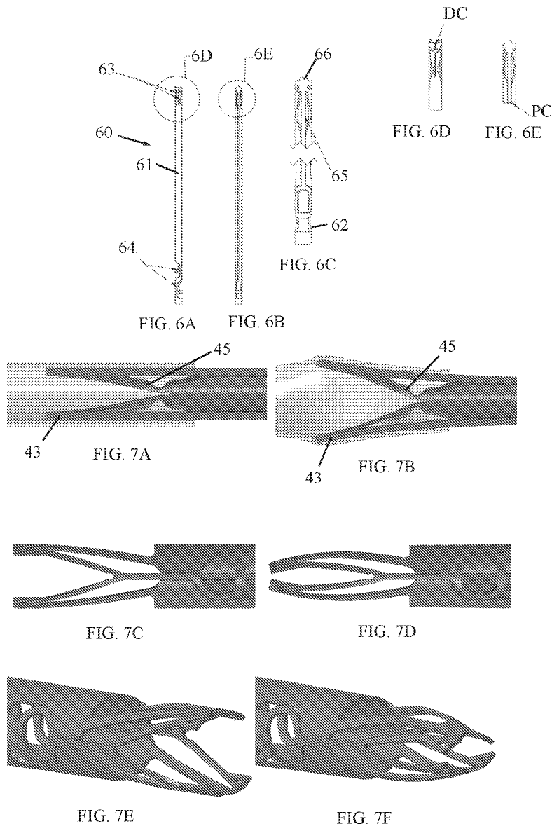

Reference is now made to FIGS. 6A-6E, which illustrate a manipulating device 60, in accordance with another embodiment of the present invention. As in other embodiments, device 60 includes a hollow tube 61 having a tube wall 62 formed with distal cutouts (DC, FIG. 6D) that define one or more deformable members 63 and proximal cutouts (PC, FIG. 6E) that define an actuator 64 in the tube wall 62 connected to deformable members 63. The deformable members 63 may be connected by linkage members 65 defined by other cutouts formed in tube wall 62. At least one of the linkage members 65 is connected to actuator 64, as seen in FIG. 6B.

This embodiment includes one or more than one actuator 64, and each actuator 64 is formed from the tube wall. Each deformable member 63 is separately controlled by its own dedicated actuator 64. The deformable members 63 may end in a distal cap member 66. The deformable members 63 may expand outwards when actuated by the actuators 64.

Optionally, the tube 61 may be covered with an external sleeve (rigid or flexible sleeve, which may optionally be formed with cutouts), which may increase of the jaws grasping force. For example, the deformable members 63 of manipulating device 60 may be deployed outwards by pulling the actuator 64. While the deformable members 63 are expanded outwards, a tissue may be engaged by the deformable members 63, which serve as jaws to grasp the tissue. Afterwards, the actuator 64 may be released and the tissue is held tight by the deformable members 63 due to the elasticity or super-elasticity of the tube material. The external sleeve may be moved over part or all of the deformable members 63 to increase the grasping force.

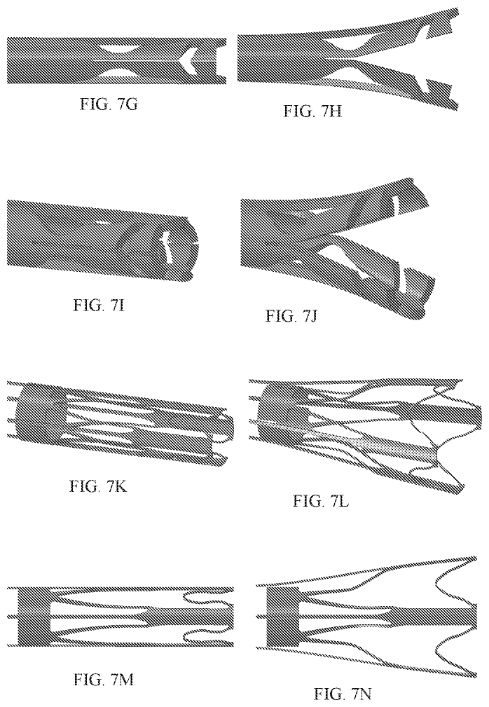

Reference is now made to FIGS. 7A-7B, 7C-7D, 7E-7F, 7G-7H, 7I-7J, 7K-7L and 7M-7N, which illustrate orientations of the deformable members and linkage members of manipulating devices of embodiments of the invention, respectively, before and after actuation of the actuator. It is seen that there is a wide variety of possibilities of deformations and the devices may have many applications in medical procedures.

The embodiment of FIGS. 7A-7B is similar to device 40 and can be designed to be normally closed and then opened when the jaws deform outwards (such as for dilation) or normally open and then closed when the jaws deform inwards (such as for grasping or cutting).

The embodiments of FIGS. 7C-7F are normally open and then closed when the jaws deform inwards by pulling the linkage members (such as for grasping, cutting or anchoring).

The embodiment of FIGS. 7G-7J is similar to device 60 and can be designed to be normally closed and then opened when the jaws deform outwards (such as for dilation).

The embodiment of FIGS. 7K-7N is similar to device 40 or 60. The working tip may be used as a retractor to provide a safe working zone.

In any of the embodiments of the invention, the tube may be used as a sensing device to measure the force required to pull the actuator and/or the force required to deform the deformable member. The sensing may be done inside or outside the tube, or inside or outside the patient. For example, the force may be measured by one or more strain gauges mounted on the device or by optical measurement of the strain induced in the tube. Possible applications include improved and more accurate placement of an epidural needle or a pedicle screw.

What is claimed is:

1. A manipulating device comprising:
a hollow tube having a tube wall formed with distal cutouts that define at least one deformable member in said tube wall and proximal cutouts that define an actuator in said tube wall connected to said at least one deformable member, wherein movement of said actuator deforms said at least one deformable member from a first orientation to a second orientation, and
wherein said at least one deformable member comprises jaws, wherein in the second orientation said jaws are separated from each other by a larger gap than in the first orientation.

2. The manipulating device according to claim 1, wherein said at least one deformable member comprises a stationary jaw and a movable jaw, wherein in the second orientation said jaws are separated from each other by a larger gap than in the first orientation.

3. The manipulating device according to claim 1, wherein said at least one deformable member comprises a plurality of deformable members connected by linkage members defined by other cutouts formed in said tube wall.

4. The manipulating device according to claim 1, wherein at least one of said deformable members has a different thicknesses than other one of said deformable members.

5. The manipulating device according to claim 1, wherein at least one of said deformable members has different thicknesses at different portions thereof.

6. The manipulating device according to claim 1, wherein said at least one deformable member expands outwards in the second orientation.

7. The manipulating device according to claim 1, wherein said at least one deformable member expands inwards in the second orientation.

8. The manipulating device according to claim 1, wherein said at least one deformable member comprises a deformable ring.

9. The manipulating device according to claim 1, wherein said at least one deformable member in the second orientation forms a retractor.

10. The manipulating device according to claim 1, wherein said at least one deformable member comprises cutting surfaces.

11. The manipulating device according to claim 1, wherein said at least one deformable member comprises a grasping element.

12. The manipulating device according to claim 1, wherein said at least one deformable member comprises an electrode.

13. The manipulating device according to claim 1, wherein said actuator comprises a handle interface portion.

14. The manipulating device according to claim 1, wherein said tube wall has an additional cutout in which said actuator is movable.

15. A method for making a manipulating device comprising:
forming at least one deformable member and an actuator connected to said at least one deformable member by making cutouts in a wall of a hollow tube, said cutouts defining outlines of said at least one deformable member and said actuator, and wherein said at least one deformable member comprises jaws, wherein in the second orientation said jaws are separated from each other by a larger gap than in the first orientation.
